(19) United States
(12) Patent Application Publication
(10) Pub. No.: US 2024/0164454 A1
(43) Pub. Date: May 23, 2024

(54) VAPOR INHALATION DEVICE INCLUDING A PIVOT FOR ROTATING

(71) Applicant: Pangea Proper LLC, Sheridan, WY (US)

(72) Inventor: Michael John Bosworth Roberts, West Hollywood, CA (US)

(21) Appl. No.: 17/990,529

(22) Filed: Nov. 18, 2022

(51) Int. Cl. A24F 40/60; A24F 40/10; A24F 40/40; A24F 40/90; A61M 11/04; F16C 11/04

(52) U.S. Cl. CPC A24F 40/60; A24F 40/10; A24F 40/40; A24F 40/90; A61M 11/042; F16C 11/04

(57) ABSTRACT

A vapor inhalation device may include a housing. The housing may contain a reservoir that holds liquid. A heating element that heats the liquid from the reservoir to vapor may be located in the housing. The housing may include a power supply that provides power to the heating element. The housing may include a port configured to be engaged by a mouth of a user. A fluid circuit may be configured to convey vapor formed from the liquid from the reservoir to the port for inhalation through the mouth of the user engaged with the port. The vapor inhalation device may include a pivot configured to be engaged by at least one finger of the user. The pivot may formed such that engagement by the at least one finger of the user forms an axis of rotation for the housing about which the housing is rotatable.

### FIELD OF THE DISCLOSURE

This disclosure relates to vapor inhalation devices having enhanced stress relieving and entertainment capabilities.

### BACKGROUND

People have been smoking various plants and herbs for centuries in order to reduce stress, relax, and have fun. In the last few decades, various health issues have been linked to smoking. Recently, as an alternative to smoking, inhaling vapor mixed with various additives from a vapor inhalation devices has become popular. These devices come in various shapes and sizes, and have the sole purpose of creating vapor for a user to inhale. As such, any benefit they provide comes from the act of inhaling vapor itself.

The present disclosure seeks to increase the stress relieving and entertainment capabilities of vapor inhalation devices. The present disclosure is directed to a vapor inhalation device that includes a pivot that is configured to be engaged by at least one finger of a user, such that engagement by the at least one finger forms an axis of rotation for the device.

### SUMMARY

One or more aspects of the present disclosure relate to an apparatus having a housing. The housing may include a reservoir that holds a liquid. The housing may also include a heating element that heats the liquid from the reservoir to vapor. The housing may have a power supply that provides power to the heating element. The housing may include a port configured to be engaged by a mouth of a user. A fluid circuit may be included that is configured to convey vapor formed from the liquid from the reservoir to the port for inhalation through the mouth of the user engaged with the port. The housing may include a pivot configured to be engaged by at least one finger of the user. The pivot may be formed such that engagement by the at least one finger of the user forms an axis of rotation for the housing about which the housing is rotatable.

In some aspects, the pivot may include a receptacle and a bearing may be positioned within the receptacle.

In some aspects, the bearing may include a sleeve.

In some aspects, the bearing may include a horizontal surface configured to engage at least one finger.

In some aspects, a surface that is engaged by at least one finger may remain fixed rotationally while the housing rotates around the surface.

In some aspects, the apparatus may include a port electrically coupled to a power supply, the port may be configured to convey power to the power supply for recharging.

In some aspects, the housing may include a curved upper surface, a curved lower surface, and a side wall between the upper surface and the lower surface.

In some aspects, the side wall, curved upper surface, or curved lower surface may include a transparent window. In some aspects, the transparent window may be located adjacent the reservoir.

In some aspects, the apparatus may include a power level indicator configured to reflect an amount of power in the power supply.

In some aspects, the power supply and the reservoir may be positioned at a distance from the pivot to maximize a moment of inertia.

In some aspects, the power supply may be positioned farther from the pivot than the reservoir.

In some aspects, the reservoir may be positioned farther from the pivot than the power supply.

In some aspects, the housing may be formed of one or more plastic, metal, and glass.

In some aspects, the reservoir may be removable.

In some aspects, the apparatus may include a temperature sensor.

In some aspects, the apparatus may include a button to initiate heating of the heating element.

In some aspects, the housing may include a first end, a second end, a straight side wall, and a curved sidewall.

In some aspects, the housing may taper from the first end towards the second end.

Another aspect of the disclosure relates to an apparatus having a housing, the housing may include a first end, a second end, a straight side wall, and curved sidewall. The housing may also include a reservoir configured for receiving one or more liquid. The housing may have a heating element for heating the one or more liquid. The housing may include a power supply. The housing may also have a receptacle having a rotatable sleeve, the receptacle being configured for receiving a finger. In some aspects, the housing may be configured to rotate around a finger positioned in the receptacle.

These and other features and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a top view of a vapor inhalation device, in accordance with one or more implementations.

FIG. 2 illustrates a bottom view of the vapor inhalation device of FIG. 1, in accordance with one or more implementations.

FIG. 3 illustrates a side view of the vapor inhalation device of FIG. 1, in accordance with one or more implementations.

FIG. 4a and FIG. 4b illustrate cross-sectional views of the vapor inhalation device of FIG. 1, in accordance with one or more implementations.

FIG. 5 illustrates the vapor inhalation device of FIG. 1 in use, in accordance with one or more implementations.

### DETAILED DESCRIPTION

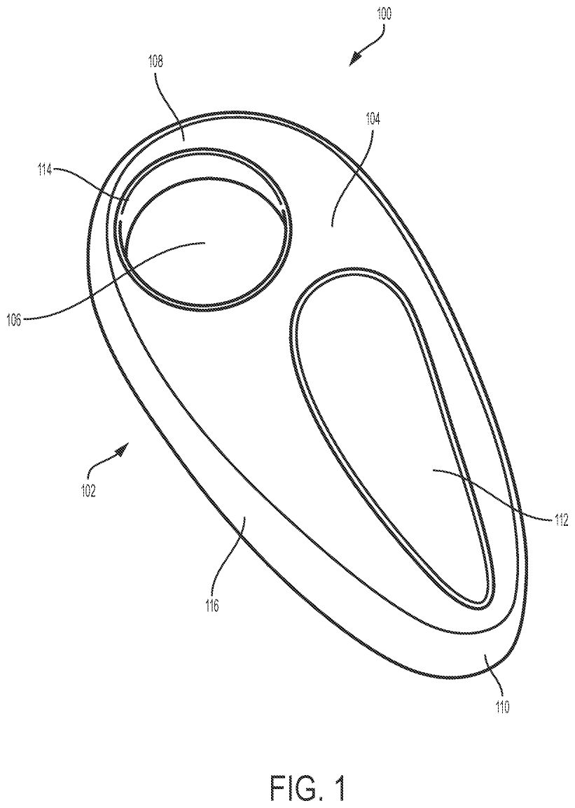

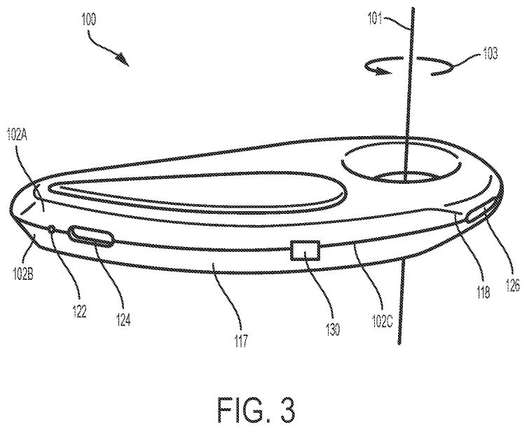

FIG. 1 illustrates a top view of a vapor inhalation device 100, in accordance with one or more implementations. Vapor inhalation device 100 includes a housing 102. The housing 102 may be formed of one or more plastic, metal, and glass. In some aspects, the housing 102 may be formed of two pieces, such as an upper half 102A and a lower half 102B as illustrated in FIG. 3. The upper half 102A and the lower half 102B may be joined together at seam 102C to encase or hold components of the vapor inhalation device 100 therein. The upper half 102A and the lower half 102B may be joined by ultrasonic welding, adhesives, heat treatment, or other methods to connect the upper half 102A and the lower half 102B.

The housing 102 may include a curved upper surface 104 and a curved lower surface 105. In some aspects, the curvature of the upper surface 104 and the lower surface 105 may be similar such that the housing 102 has a consistent thickness throughout. In other aspects, the curvature of the upper surface 104 and the lower surface 105 may be different such that the housing 102 is thicker in some areas relative to other areas. The curved upper surface 104 and the curved lower surface 105 may be configured to accommodate a curve of a palm of a user. The housing 102 may also include a first end 108 and a second end 110 opposite the first end 108. The first end 108 and the second end 110 may be rounded. The housing 102 may taper from the first end 108 towards the second end 110.

A pad 112 may be positioned on the upper surface 104. The pad may be formed of a material different than housing 102. The pad 112 may be adhered or otherwise affixed to the upper surface 104 of the housing 102. The pad 112 may be formed of a material having a greater friction coefficient than the housing to reduce the likelihood of a user dropping the vapor inhalation device 100. The pad 112 may also be formed of a compressible material. In some aspects, the pad 112 may include a first end 113 and a second end. The pad may taper from the first end 113 having a larger rounded portion to the second end 115. In other aspects, the pad 112 may include a circular, oval, square, rectangular, or polygonal shape. In some aspects, the pad 112 may take the shape of a character, mascot, or wording.

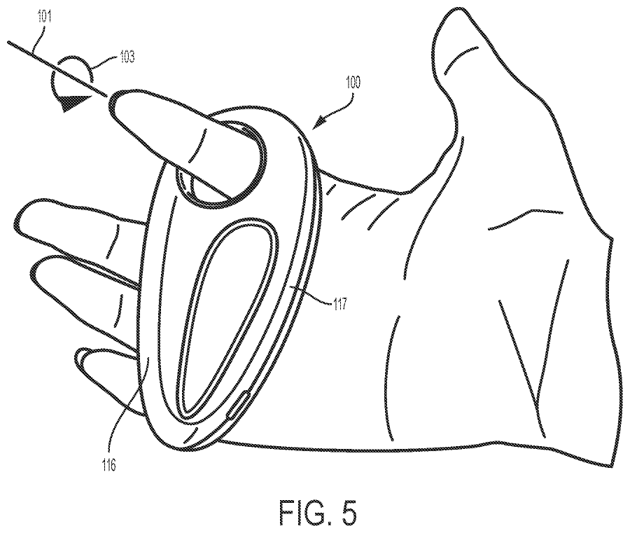

The housing 102 may include a pivot 106 configured to be engaged by at least one finger of a user. The pivot 106 may be located adjacent the first end 108 of the housing 102. The pivot 106 may be offset from a longitudinal axis extending from the first end 108 to the second end. The pivot 106 may be formed such that engagement by at least one finger of a user forms an axis of rotation 101 for the housing 102 about which the housing 102 is rotatable (as indicated by arrow 103). As such, when a user inserts a finger into pivot 106, as illustrated in FIG. 5, the user may spin the vapor inhalation device 100 in a circular motion as indicated by arrow 103 around axis 101.

In some aspects, the pivot 106 may include a hole or receptacle into which a user may insert a finger. The hole or receptable may take the form of a circle, oval, ellipse, rectangle, square, or polygon. In some aspects, the pivot 106 may include a bearing 114 located within the hole or receptacle. The bearing may be formed of a plastic, metal, or combination thereof. The bearing may include a surface that is configured to remain stationary when in contact with a finger of a user while the housing 102 rotates. The surface may be engaged by a portion of at least one finger and remain fixed rotationally while the housing 102 rotates around the surface.

In one aspect, the bearing 114 may take the form of a sleeve positioned within pivot 106. The sleeve may include a first lip that engages the upper surface 104 and a second lip that engages the lower surface 105. The sleeve may be configured to rotate freely within pivot 106.

In another aspect, the bearing 114 may include an outer rim that contacts an inner surface of pivot 106 and remains stationary thereto. The bearing 114 may include a central cylinder or horizontal surface that freely rotates within the outer rim. In such a configuration, a user may grasp the central cylinder or horizontal surface on opposite sides with a thumb and forefinger, and rotate the housing 102 around the pivot 106.

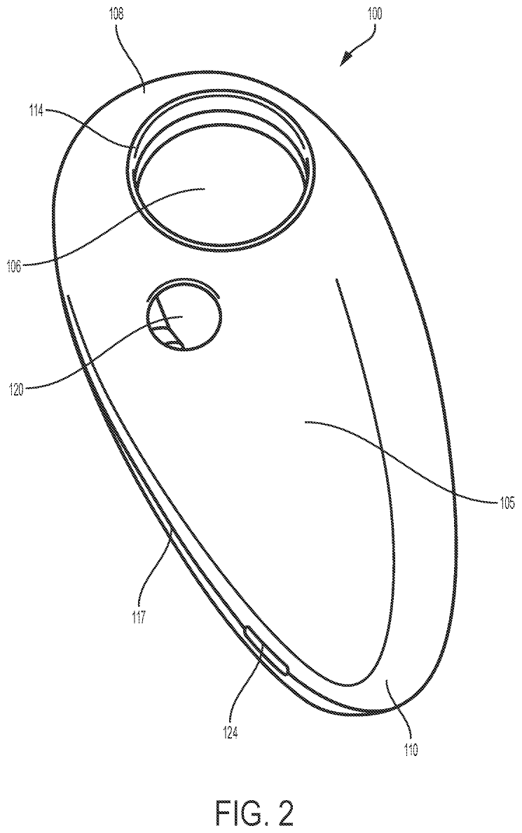

As seen in FIG. 1, the housing 102 may also include a sidewall extending between the upper surface 104 and the lower surface 105. A first edge 116 of the sidewall may include a surface that curves from the upper surface 104 to the lower surface 105. The first edge 116 allows for the vapor inhalation device 100 to be comfortably gripped by the pinky finger, ring finger, and middle finger of a user while the index finger is inserted through pivot 106. A second edge 117, as seen in FIG. 2, FIG. 3, and FIG. 5 may include a surface that is substantially straight extending from the upper surface 104 to the lower surface 105. The second edge 117 is configured to rest comfortably in the palm of a user when vapor inhalation device 100 is gripped.

FIG. 2 illustrates a bottom view of vapor inhalation device 100. The lower surface 105 may include a transparent window 120. The transparent window 120 may be adjacent the reservoir located within housing 102. The transparent window 120 is configured so that a user may visually inspect a fluid within the reservoir. In some aspects, a transparent window may be located on the sidewall, or along the upper surface 104 of housing 102.

The second edge 117, as seen in both FIG. 2 and FIG. 3, may contain a power level indicator 122, a power charging port 124, and a vapor port 126. The power level indicator 122 may be configured to indicate when the vapor inhalation device 100 is turned on or off. Additionally, the power indicator 122 may provide an indication of a power level in a power supply located with the housing 102. The power level indicator 122 may be configured to reflect an amount of power in the power supply. The power indicator 122 may take the form of one or more light, such as an LED having various colors. In some aspects, a first color, such as green, may indicate that the vapor inhalation device 100 is on and ready for use. A second color, such as red, may indicate that the power source is low and needs recharging. A third color, such as blue, may indicate that the power charging port 124 is in use and the power supply is currently charging.

In some aspects, the power charging port 124 may allow for charging of the power supply from an external source of electricity. In some aspects, the power charging port 124 may be configured as a USB port, lightning port, micro USB port, USB type-C port, USB type-C port, pin port, AC port, or DC port. The power charging port 124 may be electrically coupled to the power supply and be configured to convey power to the power supply for recharging. In some aspects, the power supply may be recharged by induction.

The vapor port 126 may be located near the first end 108 of the housing 102 along a portion of the sidewall. The vapor port 126 may be located near the first end 108 to enable inhalation through a mouth of a user while the vapor inhalation device 100 is being gripped through pivot 106 by a finger of the user. In some aspects, the vapor port 126 may be located on the second edge 117. In other aspects, the vapor port 126 may be located on the first edge 116 near a transition zone 118 where the first edge 116 and the second edge 117 meet. The vapor port 126 may include an elongated opening or slit having an axis that is congruent with seam 102C.

FIG. 3 also illustrates a button 130 located on the second edge 117 of the sidewall of the housing 102. Button 130 may be configured to initiate heating of the heating element in order to produce vapor as discussed below. In some aspects, button 130 may be positioned on the upper surface 104, lower surface 105, or first edge 116 of housing 102.

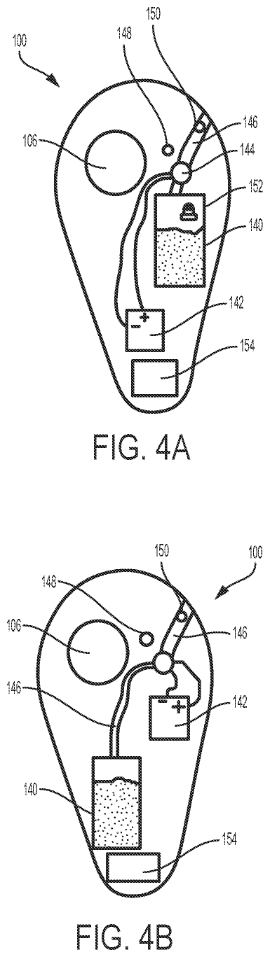

FIG. 4A and FIG. 4B illustrate cross-sectional views of vapor inhalation device 100 showing schematic representations of inner components. The inner components are positioned at a distance from the pivot 106 such that a moment of inertia is maximized. This allows for the vapor inhalation device 100 to rotate around a finger of a user easily and without interruption.

The vapor inhalation device 100 includes a reservoir 140 configured for holding a liquid. The liquid may include one or more flavoring, drug, or additive. A fluid circuit 146 is configured to convey vapor formed from the liquid from the reservoir to the vapor port 126 for inhalation through a mouth of a user. The fluid circuit 146 may include one or more conduit, channel, and wick. In some aspects, the fluid circuit 146 may be formed by a portion of the upper half 102A mating with a portion of the lower half 102B.

FIG. 4A shows the power supply 142 being positioned farther from the pivot 106 than the reservoir 140. In some aspects, power supply 142 make take the form of a rechargeable battery or capacitor. FIG. 4B illustrates the reservoir 140 being positioned farther from the pivot 106 than the power supply 142. In these configurations, weight 154 may be positioned opposite the pivot 106. The mass of the weight 154 and/or the mass of the power supply 142 may maintain momentum of the vapor inhalation device 100 as the amount of liquid in the reservoir 140 reduces over time. Maintaining momentum of the vapor inhalation device 100 may facilitate rotation of the vapor inhalation device 100 around an axis. The reservoir 140, power supply 142, and weight 154 may carry the most mass of vapor inhalation device 100. Reservoir 140, power supply 142, and weight 154 may be positioned such that weight is evenly distributed throughout. In some aspects, the reservoir 140 may be removable and the housing 102 may include one or more door, flap, or opening to access the reservoir 140.

A heating element 144 may be configured to heat the liquid from the reservoir 140 to vapor. The heating element 144 may be heated upon receiving power from power supply 142. In some aspects, the heating element 144 may be positioned along fluid circuit 146 such that the heating element 144 heats liquid from the reservoir 140 though conduction. In other aspects, the heating element 144 may be positioned along fluid circuit 146 such that the heating element 144 heats liquid from the reservoir through convection. A temperature sensor 148 may be positioned along a portion of fluid circuit 146 or adjacent the heating element 144.

During use, a user may engage the vapor port 126 by the mouth and inhale. The vapor inhalation device 100 may be configured to automatically initiate heating of the liquid from the reservoir 140 to vapor while the user inhales. In this configuration, the vapor inhalation device 100 may include a flow sensor 150 that initiates heating of the heating element 144 upon detection of a flow of air or fluid through a portion of fluid circuit 146. In some aspects, the flow sensor 150 may initiate heating of the heating element 144 based on detection of inhalation at the a vapor port 126. The flow sensor 150 may take the form of a pressure sensor, differential pressure flow sensor, thermal mass flow sensor, velocity flow sensor, volumetric flow sensor, inferential flow sensor, or other sensor that allows for the detection of air or fluid flow through a portion of fluid circuit 146.

In another aspect, a user may depress button 130 while simultaneously inhaling to initiate vapor production. When not using the vapor inhalation device 100 to produce vapor, a user may place at least one finger into pivot and rotate housing 102 to reduce stress and entertain themselves. When the device is in use in this fashion, the user's desire to inhale vapor may be decreased in favor of using the pivot functionality.

Vapor inhalation device 100 may also include one or more light 152 configured to illuminate a portion of vapor inhalation device 100 during use. In some aspects, the one or more light 152 may be located adjacent reservoir 140 so as to illuminate the contents of the reservoir 140, making the contents visible at transparent window 120. As such, when vapor is being produced and delivered to vapor port 126, transparent window 120 may be illuminated by the one or more light 152. In one aspect, the one or light 152 may take the form of an incandescent bulb or LED. The one or more light 152 may be capable of producing a plurality of colors.

FIG. 5 illustrates the vapor inhalation device 100 being held by a user in a manner that allows for spinning of the vapor inhalation device 100 around an axis 101 created by an index finger inserted into pivot 106. A user may spin the device around axis 101 as indicated by arrow 103. When the user desires to utilize vapor inhalation device 100 for vapor production, the user may simply close their fingers to quickly catch the vapor inhalation device 100 between the pinky finger, ring finger, middle finger, and the palm. The user simply raises the vapor inhalation device 100 such that the vapor port 126 contacts the mouth. When finished, the user may again spin the vapor inhalation device 100 in a manner to reduce stress and entertain.

It is noted that terms such as “posterior”, “anterior”, “forward”, “rearward”, “front”, “rear”, “upper”, “lower”, “distal”, “proximal”, “left”, and/or “right” may refer herein to anatomical directions when considering the device in an as used position and/or based on their described use. The use of these terms with various components should therefore be easily understood by a person skilled in the art as related to orientation, direction, and/or disposition. Further, some directions may be specifically defined herein and shown in the figures.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. An apparatus, comprising:
a housing, the housing containing:
a reservoir that holds liquid;
a heating element that heats the liquid from the reservoir to vapor;
a power supply that provides power to the heating element;
a port configured to be engaged by a mouth of a user;
a fluid circuit configured to convey vapor formed from the liquid from the reservoir to the port for inhalation through the mouth of the user engaged with the port; and
a pivot configured to be engaged by at least one finger of the user,
wherein the pivot is formed such that engagement by the at least one finger of the user forms an axis of rotation for the housing about which the housing is rotatable.

2. The apparatus of claim 1, wherein the pivot comprises a receptacle and a bearing is positioned within the receptacle.

3. The apparatus of claim 2, wherein the bearing is a sleeve.

4. The apparatus of claim 1, wherein the bearing includes a horizontal surface configured to engage the at least one finger.

5. The apparatus of claim 1, wherein a surface that is engaged by the at least one finger remains fixed rotationally and the housing rotates around the surface.

6. The apparatus of claim 1, further comprising:
a port electrically coupled to the power supply, the port being configured to convey power to the power supply for recharging.

7. The apparatus of claim 1, wherein the housing includes a curved upper surface, a curved lower surface, and a side wall between the upper surface and the lower surface.

8. The apparatus of claim 5, wherein the side wall, curved upper surface, or curved lower surface includes a transparent window.

9. The apparatus of claim 6, wherein the transparent window is adjacent the reservoir.

10. The apparatus of claim 1, further comprising:
a power level indicator configured to reflect an amount of power in the power supply.

11. The apparatus of claim 1, wherein the power supply and the reservoir are positioned at a distance from the pivot to maximize a moment of inertia.

12. The apparatus of claim 1, wherein the power supply is positioned farther from the pivot than the reservoir.

13. The apparatus of claim 1, wherein the reservoir is positioned farther from the pivot than the power supply.

14. The apparatus of claim 1, wherein the housing is formed of one or more plastic, metal, and glass.

15. The apparatus of claim 1, wherein the reservoir is removable.

16. The apparatus of claim 1, further comprising:
a temperature sensor.

17. The apparatus of claim 1, further comprising:
a button to initiate heating of the heating element.

18. The apparatus of claim 1, wherein the housing includes a first end, a second end, a straight side wall, and a curved sidewall.

19. The apparatus of claim 17, wherein the housing tapers from the first end towards the second end.

20. An apparatus, comprising:
a housing, the housing having a first end and a second end, a straight side wall, and curved sidewall, the housing comprising:
a reservoir for receiving one or more liquid;
a heating element for heating the one or more liquid;
a power supply; and
a receptacle having a rotatable sleeve, the receptacle being configured for receiving a finger;
wherein, the housing is configured to rotate around a finger positioned in the receptacle.
